(19) United States
(12) Patent Application Publication
(10) Pub. No.: US 2026/0144608 A1
(43) Pub. Date: May 28, 2026

(54) TACTILE FEEDBACK SENSOR

(71) Applicant: ARIZONA BOARD OF REGENTS ON BEHALF OF THE UNIVERSITY OF ARIZONA, Tucson, AZ (US)

(72) Inventors: Dema Govalla, Las Vegas, NV (US); Jerzy Rozenblit, Tucson, AZ (US)

(73) Assignee: ARIZONA BOARD OF REGENTS ON BEHALF OF THE UNIVERSITY OF ARIZONA, Tucson, AZ (US)

(21) Appl. No.: 19/400,827

(22) Filed: Nov. 25, 2025

Related U.S. Application Data
(60) Provisional application No. 63/725,202, filed on Nov. 26, 2024.

(51) Int. Cl. A61B 34/00; A61B 34/30; B25J 19/02; G01L 1/22

(52) U.S. Cl. CPC A61B 34/76; A61B 34/30; B25J 19/02; G01L 1/22

(57) ABSTRACT

This invention provides systems and methods for tactile perception in robotic assisted minimally invasive surgery (RAMIS), focusing on deformation and texture detection. The system integrates microelectromechanical systems (MEMS) sensors and/or force-sensitive resistor (FSR) sensors attached to a thoracic grasper instrument and is compatible with the da Vinci MIS surgical system and other robotic surgical systems. The systems further utilize algorithms able to accurately classify objects with varying softness and roughness into corresponding deformation or texture labels. By designing and implementing a sensor-based system which comprises feature extraction and a recognition module, the surgeon can detect texture (the roughness of a biological organ on its surface) and deformation (tissue hardness/softness).

### CROSS-REFERENCE TO RELATED APPLICATIONS

This application claims priority from U.S. Provisional Patent Application No. 63/725,202, filed Nov. 26, 2024, which is incorporated by reference herein to the extent that there is no inconsistency with the present disclosure.

### BACKGROUND OF THE INVENTION

Minimally invasive surgery (MIS) typically uses small incisions, thin instruments, and an endoscopic camera. MIS is classified into standard laparoscopy, endoscopic, and robot-assisted surgery [1]. Robot-assisted minimally invasive surgery (RAMIS) is a method that is often preferred over conventional open surgery due to several advantages. Benefits of RAMIS include reduced postoperative pain, faster recovery time, shorter hospital stays, lower risk of surgical site infections, and a reduced learning curve for surgical trainees [2]. However, a considerable problem in RAMIS is the lack of tactile feedback and palpation ability when performing surgery, which can lead to tissue or organ damage or trauma [3]. Palpation includes the surgeon's ability to examine the texture or deformation of an organ during surgery [4], and is a procedure surgeons exploit regularly and naturally in traditional (open) surgery. For example, palpation is used to locate blood vessels hidden beneath opaque tissues [5].

RAMIS emerged in 1985 with the Puma 560 robot [11], offering various advantages such as three-dimensional vision, greater dexterity, improved mobility, and reduced tremor for the operator [1]. Since then, other surgical systems such as the ZEUS and the da Vinci Surgical System are now the most complete and developed robotic platforms for use with RAMIS. Both systems are implemented for procedures such as gastrointestinal, adrenalectomy, colectomy, thyroid, and hysterectomy surgeries [12]. However, robotic surgical systems are limited in their ability to mimic tactile sensations during surgeries. Researchers have been advocating for tactile perception methods in order to obtain organic structures and environmental information for surgeons during robotic surgery. For example, the use of tactile sensors to perceive an organ or tissue's hardness or texture has been proposed by Liu et al. and Govalla et al. [13] [14]. Significant advances in tactile sensor designs have involved attaching piezoresistive, piezoelectric, capacitive, or optical sensors on the grasper portion of a surgical tool [3]. Tactile sensors can be divided into single-point and tactile array sensors. Recent research have focused on using tactile array sensors because they can cover a wider area and capture the tactile information for an organ or tissue from multiple dimensions. By doing so, the tactile array sensor can achieve a high spatial resolution of touch [2].

Previous methods of tactile array sensors include using a 4 mm×15 mm capacitive-based tactile array connected to a sensing instrument [15]. The sensor measures pressure changes and displays them on a contour mapping to detect tumors via a visualization software. Another research team used a three-dimensional force sensor based on a resistive sensing method that detects the interaction forces during tissue palpation [16]. A separate research group developed a self-powered, high-resolution, and pressure-sensitive sensor based on single-electrode triboelectric generators that enable real-time tactile mapping [17]. Additionally, Li et al. created a high-sensitivity optical tactile sensor array based on fiber Bragg grating (FBG), where the optical sensor allows for exploring and localizing tissue abnormalities during palpation [18].

Other methods include commercial devices such as the BioTac SP tactile sensor manufactured by Syntouch, where the sensor consists of a 12 mm×11 mm matrix in which two electrodes are shown to the surgeon in an image using the mean value of the eight nearest neighbors [19] [20]. To overcome constraints such as lack of sterility, lack of autoclavability, and utilization of numerous feedback wires, Naidu et al. [21] created a capacitive tactile array sensor that attaches directly to a surgical robot for palpation in RAMIS.

However, conventional systems developed from past research are higher in cost and only determine one physical characteristic for tactile perception. Accordingly, what is needed is an improved, low-cost tactile sensor and methods capable of determining both the deformation and texture types of an organ or tissue structure.

### BRIEF SUMMARY OF THE INVENTION

The present invention provides a sensor and methods capable of determining haptic and tactile characteristics of an object, including but not limited to an organ, tissue, anatomical structure, or combinations thereof, of a patient. By providing detailed tactile feedback, the sensor system of the present invention allows an operator to determine characteristics, such as structural deformations, hardness, texture, vibrations, localized shape, slipperiness/stickiness, and/or changes in consistency of the object. In an aspect of the invention, the sensor system of the present invention allows a surgeon or other medical professional to differentiate between various tissue types and texture types, improving the accuracy of surgical and medical manipulations.

In an embodiment, the present invention provides tactile sensing device comprising: a distal end and a proximal end; one or more micro-electromechanical system (MEMS) and one or more force-sensitive resistor (FSR) sensors disposed on the distal end of the device; and a microcontroller in communication with the one or more MEMS sensors and the one or more FSR sensors. As used herein, the “distal end” of the device refers to the end or portion of the device oriented toward a surface, such as a biological tissue, and used to probe or contact the surface. The “proximal end” of the device refers to the end or portion of the device oriented away from the surface or the end or portion of the device held by an operator or a control system, such as a robotic control system.

When the distal end of the device contacts a surface, the one or more MEMS sensors and the one or more FSR sensors are able to measure two or more attributes of the distal end of the device as the distal end moves across or against the surface. Preferably, the two or more measured attributes comprise force applied to the distal end, positional displacement of the distal end, linear vibration of the distal end, angular vibration of the distal end, and combinations thereof. In an embodiment, the two or more attributes indicate positional data of the distal end or forces exerted on the distal end as a result of the tactile sensing device being moved across the surface or being pressed against the surface. Electrical signals characterizing the measured attributes are generated and transmitted to the microcontroller, where the microcontroller is able to generate data of two or more characteristics of the surface from the transmitted signals.

In an embodiment, the two or more characteristics of the surface comprise hardness, texture, vibrations, localized shape, slipperiness/stickiness, changes in consistency, and combinations thereof. Preferably, the two or more characteristics of the surface comprise texture and hardness of the surface.

Micro-electromechanical systems (MEMS) are microscopic devices that incorporate electronic and mechanical components to create sensors and other mechanical devices. In an embodiment, the MEMS range in size (in an average or largest dimension) from 20 μm to 1 mm, optionally between 20 μm to 0.5 mm, or between 50 μm to 0.2 mm, and are made up of components between 1 μm and 100 μm in size, optionally between 5 μm and 80 μm, or between 10 μm and 60 μm. In an embodiment, two or more, three or more, our four or more MEMS sensors disposed on the distal end of the device. Preferably, two MEMS sensors disposed on the distal end of the device.

In an embodiment, the one or more MEMS sensors and FSR sensors comprise a magnetometer sensor, an accelerometer sensor, a gyroscope sensor, or combinations thereof. Preferably, the one or more MEMS sensors are able to measure changes in the distance and position of the distal end of the device as the distal end is moved across the surface. For example, the MEMS sensors are able to measure vibrations or how much the position of the distal end changes as the device moves across the surface. A biological tissue or other surface having greater surface roughness will likely result in the distal end undergoing greater vibrations or greater displacement.

A force-sensing resistor (FSR) is a material, including but not limited to piezoresistive devices, whose resistance changes when a force, pressure, or mechanical stress is applied to the material. In an embodiment, materials of the FSR sensor produce a decrease in electrical resistance as more physical force or pressure is applied. In an embodiment, two or more, three or more, our four or more FSR sensors disposed on the distal end of the device. Preferably, the one or more FSR sensors are able to measure force applied to the distal end of the device when the distal end is pressed against the surface. For example, less force may be exerted on the distal end as the device is pressed against a softer biological tissue or other material than a harder biological tissue or other material. In an embodiment, the FSR ranges in size (in an average or largest dimension) from 20 μm to 100 mm, optionally between 50 μm to 50 mm, between 100 μm to 10 mm, or between 50 μm to 1 mm.

In an embodiment, the microcontroller comprises a processing unit able to process the electrical signals generated from the measurements of the two or more attributes and generate texture and hardness data of the surface of the object. Preferably, the microcontroller utilizes a Reflex Fuzzy Min-Max Neural Network (RFMN) algorithm or a Time Series Classification-Learning Shapelets (TSC-LS) algorithm to generate the texture and hardness data. In a further embodiment, generating the texture and hardness data comprises identifying the texture and hardness of biological tissue with recognized or known classifications. Optionally, the recognized or known classifications are programmed or installed into the microcontroller.

In an embodiment, the tactile sensing device is a surgical instrument, preferably a surgical instrument capable of being operationally connected to a robotic surgical system, including but not limited to the da Vinci Surgical System. The ability of the tactile sensing device to be compatible with robotic surgical systems allows the present invention to be readily adopted without significant modifications to existing surgical tools.

The sensing device is used to detect and/or measure characteristics of a surface or object, including in conjunction with robotic systems. In an embodiment, the robotic system is located in a different physical location than the human operator and is operated remotely. In an embodiment, the surface or object includes, but is not limited to, agricultural products, glass, electronics, mechanical components, woven materials, and hazardous materials. Preferably, the surface or object is an organ, biological tissue, or anatomical structure of a subject, including but not limited to human organs, tissue, or anatomical structures exposed during surgical procedures or medical exams.

In an embodiment, the present invention provides a method for detecting two or more characteristics of an object, such as hardness, vibrations, texture, localized shape, slipperiness/stickiness, changes in consistency, and combinations thereof. Optionally, temperature is determined in addition to the two or more characteristics. In an embodiment, the texture and hardness of the object is detected comprising contacting the surface of the object with a tactile sensing device, wherein the tactile sensing device comprises one or more micro-electromechanical system (MEMS) sensors and one or more force-sensitive resistor (FSR) sensors disposed on a distal end of the tactile sensing device, and a microcontroller in communication with the one or more MEMS sensors and the one or more FSR sensors. The method further comprises moving the distal end of the tactile sensing device is across the surface of the object, pressing the distal end against the surface of the object, or combinations of both. This is followed by measuring two or more attributes of the distal end of the tactile sensing device as the distal end moves across or against the surface, generating electrical signals characterizing the measured attributes, and transmitting the generated signals to the microcontroller. After being received by the microcontroller, the texture and hardness data of the object is generated from the signals transmitted to the microcontroller. Optionally, the object is a biological tissue, preferably a biological tissue of a patient.

In an embodiment, the step of measuring the two or more attributes comprises moving the distal end of the tactile sensing device across the surface of the object (preferably a biological tissue) and measuring changes in distance and position of the distal end of the tactile sensing device as the distal end of the tactile sensing device is moved across the surface. Additionally, or as an alternative, the step of measuring the two or more attributes comprises pressing the distal end of the tactile sensing device against the surface of the object, thereby generating a force against the distal end of the tactile sensing device, and measuring the force applied to the distal end of the tactile sensing device. In an embodiment, the step of measuring the two or more attributes comprises measuring the force applied to the distal end of the tactile sensing device, measuring the positional displacement of the distal end of the tactile sensing device, measuring linear vibration of the distal end of the tactile sensing device, measuring angular vibration of the distal end of the tactile sensing device, and combinations thereof. In an embodiment, a human operator operates the tactile sensing device to probe the object. Optionally, the tactile sensing device is operated using a robotic surgical system.

In an embodiment, generating the data of the two or more characteristics comprises identifying the two or more characteristics of the object with recognized or known classifications. Preferably, the two or more characteristics comprise the texture and hardness of the object. Preferably, the recognized or known classifications are classifications of biological tissue. In an embodiment, identifying the two or more characteristics of the object comprises processing the signals transmitted to the microcontroller using a machine learning algorithm, Reflex Fuzzy Min-Max Neural Network (RFMN) algorithm, or a Time Series Classification-Learning Shapelets (TSC-LS) algorithm.

### BRIEF DESCRIPTION OF THE DRAWINGS

FIG. 1 shows a tactile sensing device of an embodiment of the present invention having two micro-electromechanical systems (MEMS) sensors a force-sensitive resistor (FSR) sensor positioned in the distal tip of the device.

FIG. 2 shows a tactile sensor device integrated with an arm of a robotic surgical system in an embodiment of the invention.

FIG. 3 illustrates a tactile sensor design schematic according to an embodiment of FIG. 1.

FIG. 4 shows a deformation acquisition block diagram according to an embodiment of FIG. 1.

FIG. 5 shows a texture acquisition block diagram according to an embodiment of FIG. 1.

FIG. 6 illustrates the RFMN architecture in an embodiment of the present invention.

FIG. 7 illustrates the training process of the TSC-LS algorithm in an embodiment of the present invention.

### DETAILED DESCRIPTION OF THE INVENTION

### Definitions

As used herein, “hardness” refers to the resistance of an object to deformation when physical pressure is applied. In an embodiment, detecting or measuring hardness comprises measuring the degree of deformation of either the object being touched and/or the surface of the sensor when a force is applied as well as measuring the magnitude of the applied force.

As used herein, “texture” refers to the smoothness or roughness of a surface. In an embodiment, detecting or measuring texture includes detecting or measuring deviations of the surface from a mean surface height. Optionally, detecting or measuring the texture includes measuring or detecting structures or features (such as microstructures) in the surface of the object and/or the distance between the structures or features, such as a pattern in the surface.

As used herein, detecting “vibrations” refers to detecting and/or measuring the amplitude (including the distance an area moves from its resting position, the speed of the movement, and the rate of change in the velocity of the vibrations) and/or frequency of vibrations, such as vibrations arising from within the object.

As used herein, “slipperiness” or “stickiness” refer to the resistance an item encounters as the item moves across the surface of the object. In an embodiment, detecting or measuring slipperiness or stickiness comprises determining or measuring the coefficient of friction of the surface. Optionally, the slipperiness of an object is affected by the presence of a lubricating or adhesive material on the surface.

“Localized shape” refers to the shape of an object at a specific location, including but not limited to a bulge, protrusion, lesion, or depression in the surface. In an embodiment, the localized shape refers to the shape of the surface at a region of the surface less than 5 mm², less than 1 mm², less than 0.5 mm², or less than 0.1 mm². In an embodiment, shape refers to the flatness or curvature of one or more regions, or the angle of two or more regions contacting each other along the surface.

“Change in consistency” refers to a change in one or more characteristics across an area of the surface. In an embodiment, change in consistency refers to a change in hardness and/or texture across the surface, such as a change in hardness or texture of body tissue in an injured area, an area of the body being manipulated or operated on, or an area containing a tumor.

### Overview

The robotic technology employed in robot-assisted minimally invasive surgery (RAMIS) allows for greater precision, flexibility, and control compared to traditional surgical methods. However, even with significant advancements in surgical practice, the lack of haptic feedback available with RAMIS is a huge setback. Unlike traditional surgery, where surgeons can feel the tissues that they are manipulating, RAMIS currently lacks haptic and tactile feedback, which can make it more challenging for surgeons to assess tissue characteristics and apply the appropriate amount of force.

Accordingly, being able to provide tactile feedback is crucial for the performance of RAMIS, especially for surgeons palpating subsurface tumors and other organ structures. However, in RAMIS there is no standardized method to obtain tactile feedback. For this reason, the present invention provides a sensor-based system which comprises feature extraction and a recognition modules. The framework contributes to a surgeon's ability to detect texture (the roughness of a biological organ on its surface) and deformation (tissue hardness/softness).

In embodiments described below, data is gathered during feature extraction from two microelectromechanical (MEMS) sensors and a force-sensitive resistor (FSR) sensor attached to surgical instrument compatible with a robotic surgical system. Digital signal processing techniques are then applied to process the acquired data. In the recognition phase, the extracted features serve as inputs for training and testing two advanced machine learning algorithms: Reflex Fuzzy Min-Max Neural Network (RFMN) and Time Series Classification-Learning Shapelets (TSC-LS). These algorithms aim to accurately classify objects with varying softness and roughness into corresponding deformation or texture labels. The examples provided below present preliminary experiments and results analyzing the performance metrics of two machine learning algorithms

Additionally, the tactile sensor and methods in embodiments of the invention are able to provide high-resolution data as input to the feature extraction phase of the research. The extracted features are used as inputs for training and testing different artificial intelligence (AI) methods, including but not limited to clustering, neural networks (NN), support vector machine (SVM), decision tree, and Knearest neighbor algorithm.

These AI-based techniques are able to produce tactile information for hardness and texture type detection [2] [22]. While previously researched AI methods are not directly related to tactile perception in RAMIS, they have been applied to similar scenarios in medical device applications. For instance, Lee and Won [23] investigated a tissue inclusion characterization method for early breast tumor identification, and presented a novel method to estimate tissue inclusion's stiffness and geometric information using a 3-D finite-element-model-based forward algorithm and a neural network-based inversion algorithm. A method based on artificial tactile sensing (ATS) and artificial neural networks (ANNs) called intraoperative thermal imaging (ITT) was developed by Sadeghi-Goughari and Mojra [24]. The algorithm included forward analysis and inverse analysis based on ANN. Zhao et al. [25] proposed an AI method for tracking a surgical instrument. The instrument shaft is tracked using line features through the random sample consensus (RANSAC) scheme. At the same time, the end-effector is depicted using special image features based on a well-trained convolutional neural network. Although previous AI works are not directly related to reasoning data from tactile sensors, they show the potential of integrating AI in RAMIS.

As describe further in the examples below, two machine learning (ML) techniques are adopted, and various metrics of each model are analyzed to advance the of tactile feedback in RAMIS.

### Example 1

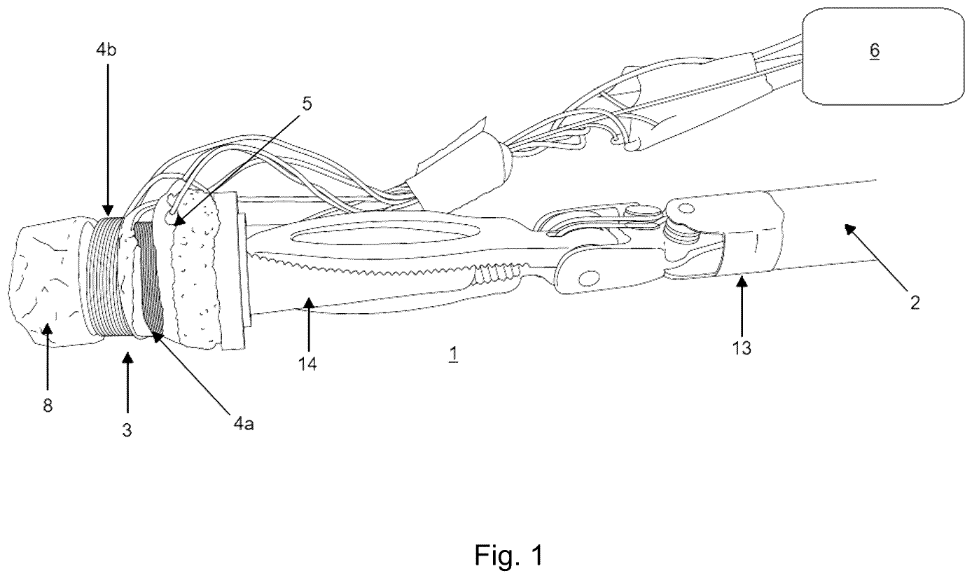

FIG. 1 illustrates a tactile sensing device 1 having a distal end 2 and a proximal end 3, where the distal end 3 is in contact with the surface of an object 8 to be examined. The sensing device 1 in this particular example comprises two micro-electromechanical systems (MEMS) sensors 4a and 4b and a force-sensitive resistor (FSR) sensor 5, although different combinations of MEMS and FSR sensors may be used. In one design, the MEMS sensors used in this example are the MLX90397 3D magnetometer sensor (4a) from Melexis and a two-in-one LSM6DSOX 3-axis accelerometer/gyroscope sensor (4b) from STMicroelectronics [6] [7]. In this embodiment, the sensors are built on top of a 3D-printed polylactic acid (PLA) base 14 attached to an EndoWrist thoracic grasper 13. Data is collected from each sensor and transmitted to a microcontroller 6.

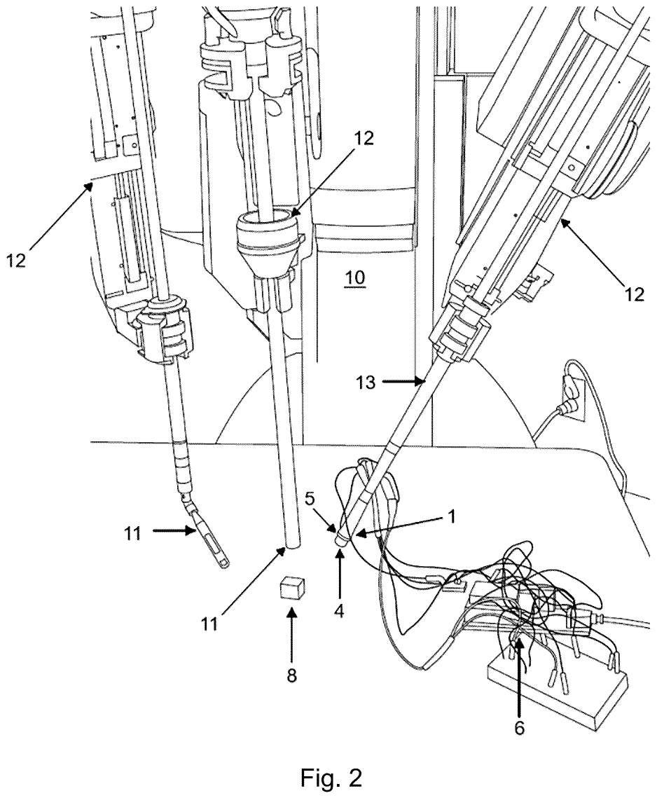

FIG. 2 shows the tactile sensing device 1 of FIG. 1 integrated with a robotic surgical system 10 having multiple arms 12. Each arm 12 of the robotic surgical system 10 can have a surgical or medical instrument 11 disposed at the tip. One arm 12 is modified to have a thoracic grasper 13 or other instrument able to hold the tactile sensing device 1. The robotic surgical system 10 uses the tactile sensing device 1 to examine the properties of the object (such as a body part of a patient). Data is collected from the MEMS 4 and FSR sensors and the data is transmitted to a microcontroller 6, where the collected data is used to monitor or direct the cutting or operation of the other surgical or medical instruments 11.

In an design, analog data is collected from each sensor using the ADC and inter-integrated circuit (I²C) pins on a microcontroller and then digital signal processing methods are used to acquire different features (attributes). The features are considered to uniquely identify the texture and deformation of a biological organ or tissue, independent of the object's orientation or scale. The features calculated to identify the hardness of the object, particularly deformations or changes in the hardness, will be variations of the magnetometer and FSR sensor data, and the features calculated to identify texture will be variations of the accelerometer/gyroscope sensor and FSR sensor data.

The extracted features are used as inputs for the system's recognition module. The recognition module can use either a Reflex Fuzzy Min-Max Neural Network (RFMN) architecture or a Time Series Classification-Learning Shapelets (TSC-LS) algorithm. RFMN can extract the features' underlying structure and employ partially supervised (hybrid) learning. RFMN has several advantages: one is its ability to handle labeled and unlabeled data simultaneously [8]. In practice, getting a fully categorized dataset for training is not feasible or the cost of categorizing all the data is high. As a result, partially supervised learning is preferred. Another advantage of RFMN involves its fast classification ability. It can simulate the reflex mechanism inspired by a human brain when solving the problem of class (label) overlaps. The reflex mechanism activates compensatory neurons whenever test data lands in a region where two or more hyperboxes (classes) overlap. The reflex mechanism makes the network quick and more accurate when classifying datasets [8].

Next, the alternative classifier chosen is a TSC-LS. TSCLS are discriminative sub-sequences of time series that best predict a target variable [9]. The algorithm is considered here because palpation applications are continuous in time, e.g., when a surgeon needs to continuously identify the texture or deformation of an organ or tissue. The advantages of TSC-LS are: a) it is a promising approach that is easily interpretable and compact (resulting in faster classification), and b) shapelets allow for the detection of shape-based similarities in subsequences [10].

### Example 2—Hardness Sensing Apparatus

A two-in-one tactile sensing device, such as generally illustrated in FIG. 1, was developed to capture the hardness and texture type of biological structures. The first sensor is designated as a hardness detection sensor (HDS) and the second sensor as a texture detection sensor (TDS). Throughout this example, the combination of these sensors is referred to as the tactile sensor design (TSD). The TSD is mounted onto a 3D-printed base and affixed to an EndoWrist thoracic grasper. The dimensions of the 3D-printed base are 10 mm×10 mm×25 mm, ensuring it fits appropriately for the intended application.

On top of the 3D-printed base, an adhesive foam strip seal is applied. This foam layer provides some softness and acts as an insulator between the components and the rigid base. Following this, a flexible force-sensitive resistor (FSR) sensor is affixed to the foam to detect pressure forces. The FSR sensor measures 4 mm in length and operates within a mid-pressure range of 10 N to 150 N. It is connected to a voltage regulator with an onboard amplifier, which amplifies the change in resistance and outputs it as an analog signal. This analog signal is then received by the ADC pins of an Arduino Uno microcontroller for further processing.

Subsequently, a double-sided adhesive rubber is placed over the FSR sensor to create a barrier between the sensor and the next component, the magnetometer integrated chip (IC) encapsulated in polydimethylsiloxane (PDMS). The magnetometer is a cost-efficient three-dimensional magnetometer used for position sensing. It operates within a wide supply voltage range (1.7V to 3.6V), consumes low power, and is suitable for space-constrained applications (2 mm×2.5 mm×0.4 mm). The chip communicates via I²C protocol and has a high-resolution magnetic range of ±50 mT. The magnetometer is encapsulated in PDMS with four wires for power supply, ground connection, and serial and clock data communication, all connected to an Arduino Uno for I²C protocol implementation.

The 3D-magnetometer aims to detect changes in distance using a magnet encapsulated in platinum-catalyzed silicone rubber. The silicone rubber used is Ecoflex 00-35, manufactured by Smooth-On, and is cured into a mold representing a fingertip-like structure at the TSD's tip. Inside the silicone rubber are two cylindrical-shaped axially magnetized neodymium magnets sized 2 mm×1 mm. These magnets capture the displacement when the rubber is palpated horizontally on a surface, aiding in detecting the hardness of organic structures.

### Example 3—Texture Sensing Apparatus

The texture detection sensor components are built onto the rest of the tactile sensor design, also as generally illustrated in FIG. 1. The first sensor considered to construct the TDS is the flexible FSR sensor. It is required to capture the pressure applied during lateral palpation of a biological structure. Additionally, the accelerometer/gyroscope sensor, created and manufactured by STMicroelectronics, features a 3D digital accelerometer, and a 3D digital gyroscope is needed to complete the TDS for texture sensing.

The accelerometer/gyroscope sensor highlights include an “always-on” experience with low power consumption for both accelerometer and gyroscope. It features a supply voltage of 1.71 V to 3.6 V, compact footprint of 2.5 mm×3 mm×0.82 mm, SPI/I2C compatibilities, high sensitive and linearity reading for standard gravity (g) and degrees per second (dps) for the accelerometer and gyroscope, respectively.

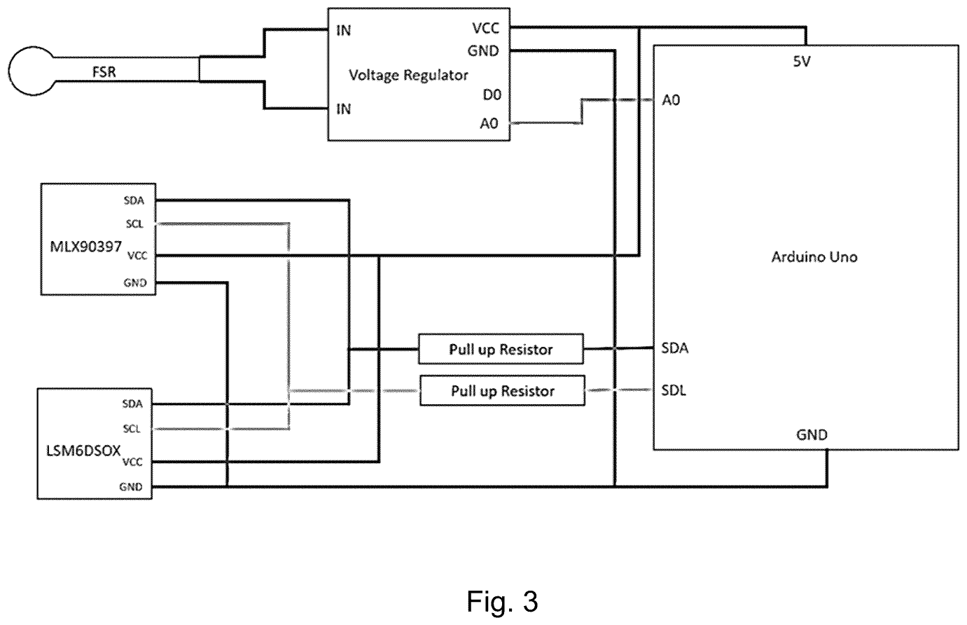

The accelerometer/gyroscope sensor is encapsulated into a PDMS structure with four wires sticking out: two wires are used for I²C communication, one for a power supply and the other for a grounding connection. The wires are connected to an Arduino Uno's I²C pins for signal processing. FIG. 3 shows the schematic of the tactile sensor design.

### Example 4—Feature Extraction Phase

The data from the tactile sensor is received at a sampling frequency of 50 Hz. Analyzing the tactile sensor's sampling data constitutes the feature extraction phase of the framework. This phase transfers raw data into numerical features that can be processed while preserving the information of the original dataset [26] [27]. For example, given a sample object, the features extracted will be unique for different texture and deformation types. Uniqueness in these features will allow the output of any classification model to differentiate between texture or deformation categories.

The features collected for deformation are measured in Force (Newton-N) and Displacement (meter-m) detected texture by the HDS, where the Force is the analog data from the flexible FSR sensor, and the Displacement is the change in position of the magnets embedded in the silicone rubber tip detected by the magnetometer IC. These attributes are considered because, given a soft object, the force generated is minimal for a given displacement compared to the force generated given a harder object. For example, it requires less effort to compress fat tissue by a displacement x than to compress muscle tissue by the same displacement x. Thus, acquiring the changes in force and displacement done by the HDS is vital to differentiate between deformation types.

Synchronously, the attributes calculated for texture are measured in Force (N), changes in Linear Acceleration in g-forces; 1g=9.81 m/s², and changes in Angular Velocity in rad/s. They will be detected by the TDS, where Force is the analog data from the flexible FSR sensor and the Linear Acceleration and Angular Velocity are captured using the accelerometer/gyroscope IC. For simplicity, Linear Acceleration and Angular Velocity will be referred to as Linear Vibration (LV) and Angular Vibration (AV), respectively. The following attributes are considered because, for a given force y, the vibrations captured by the accelerometer/gyroscope IC will vary when palpating laterally over a rough surface versus a smooth surface. Hence, the approach utilizes the differences in forces and vibrations for differentiating texture types of biological structures.

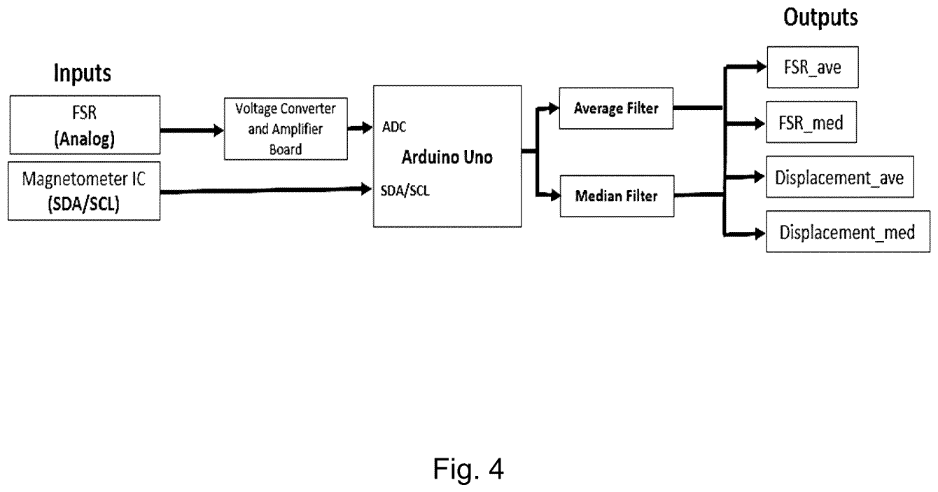

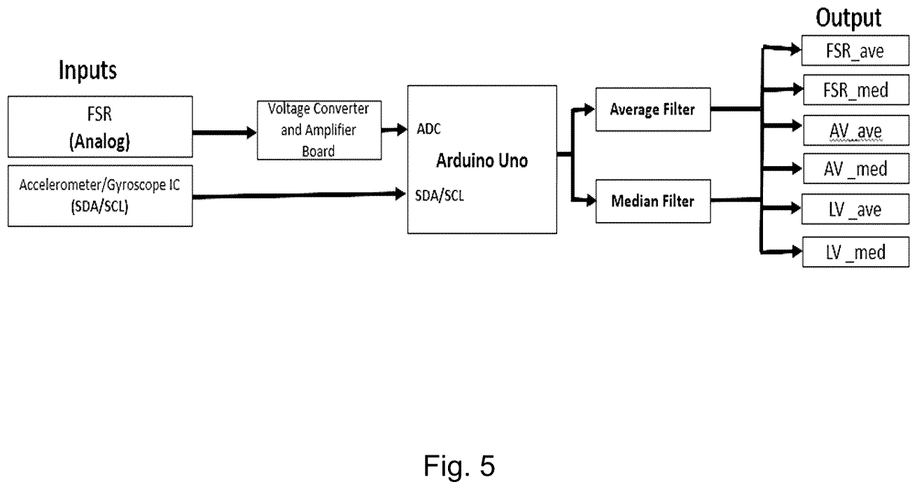

After collecting the four attributes, Force, Displacement, LV, and AV, two smoothing filters are used to reduce high-frequency noise and remove outliers without distorting the signal's integrity. The two filters used are an Average and a Median smoothing filter. Passing the attributes through both filters results in the following outputs: Forceₐᵥₑ, Force_{med}, Displacementₐᵥₑ, Displacement_{med}, LVₐᵥₑ, LV_{med}, AVₐᵥₑ, AV_{med}. Where Forceₐᵥₑ, Force_{med}, Displacementₐᵥₑ and Displacement_{med} are used for hardness detection. The flow chart for hardness feature acquisition is shown in FIG. 4. Additional, Forceₐᵥₑ, Force_{med}, LVₐᵥₑ, LV_{med}, AVₐᵥₑ and AV_{med} are used for texture detection. The flow chart for texture feature acquisition is shown in FIG. 5. These features are applied as inputs in the recognition phase of the work.

### Example 5—Recognition Phase

In this embodiment, the recognition phase comprises either an RFMN architecture or a TSC-LS algorithm. Specifically, the deformation path of the work passes four features into either algorithm for training and testing. In comparison, the texture path of the work passes six features into either algorithm for training and testing.

1) Reflex Fuzzy Min-Max Neural Network: Reflex Fuzzy Min-Max Neural Network (RFMN) is an algorithm meant to improve the original Fuzzy-Min-Max (FMM) neural network implemented by Simpson [28]. The algorithm is adapted here because it combines the operations of an artificial neural network and fuzzy set theory into a common framework, which has shown to be one of the most valuable and accurate neural networks for pattern classification [29]. RFMN can be used for supervised and partially-supervised (hybrid) learning [30]. A hybrid learning approach is beneficial because assimilating a fully labeled dataset for training may not always be feasible, or the cost of labeling all samples is not affordable [21]. Additionally, RFMN can learn in a single pass, unlike methods that use backpropagation. This advantage allows for a shorter computation time during training.

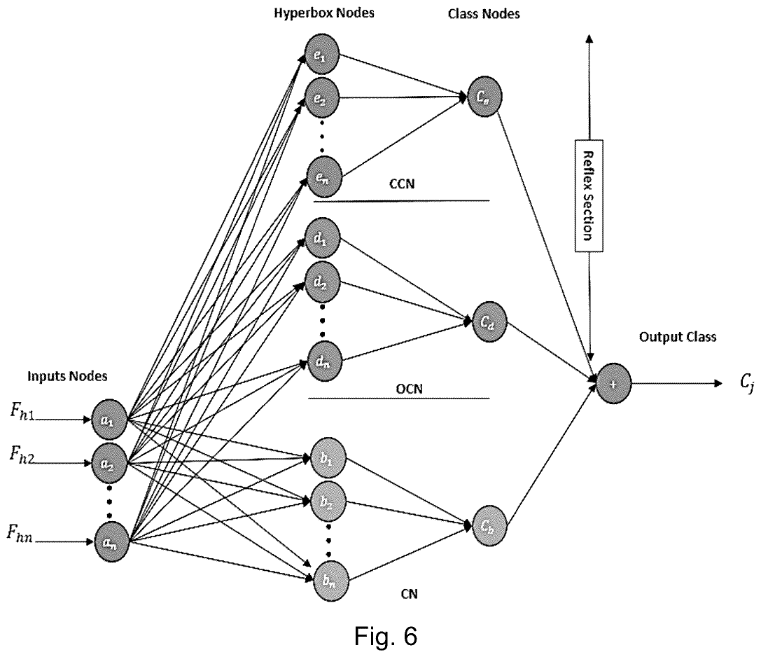

Furthermore, the detailed architecture of the RFMN is illustrated in FIG. 6 (see also Nandedkar and Biswas, “Object recognition using reflex fuzzy min-max neural network with floating neurons”, Computer Vision, Graphics and Image Processing: 5th Indian Conference, ICVGIP 2006), where the inputs are taken from a feature vector: (FV) Fₕᵢ={a₁, a₂, . . . , aₙ} (h∈1, 2, . . . , N), belonging to class Cⱼ (j∈1, 2, . . . m), where N represents the different FVs, n describes the different features in the FV and where m represents the number of classes (labels). Alternatively, a dataset {Fₕᵢ, Cⱼ} is used to train the algorithm. During training, the inputs aᵢ are given, and the network trains in a single pass and assigns every Cⱼ a membership value, as shown in FIG. 6 (Class Nodes). The fuzziness of this network arises when a hyperbox (classes) overlaps or contains each other. For instance, the network creates an overlap compensation neuron (OCN) section and a containment compensation neuron (CCN) section. The sections are formed specifically for a situation where FVs hyperbox representing different classes overlap or are contained in each other. The sections are created during training and are used when classifying unseen data during testing when ambiguity between a class category is formed. The final membership calculation considers the maximum number between all classes, max (Cⱼ), and assigns the unseen data to this label.

For the recognition section, there are two paths exiting the feature extraction section, one for deformation and another for texture. The deformation path will have inputs of a FV, Fₕᵢ, which takes Forceₐᵥₑ, Force_{med}, Displacementₐᵥₑ and Displacement_{med} as inputs and outputs a corresponding class label Cⱼ. For deformation, the class label output Cⱼ∈{Very Soft, Soft, Hard, VeryHard}. For clarification, the inputs and outputs used for training are shown below:

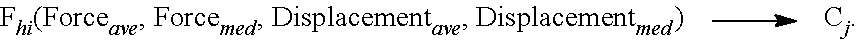

Additionally, the texture's path will have inputs of an FV, Fₕᵢ consisting of its inputs—Forceₐᵥₑ, Force_{med}, LVₐᵥₑ, LV_{med}, AVₐᵥₑ and AV_{med}. The output of the texture path will be a classification class label Cⱼ, where Cⱼ E {Very Smooth, Smooth, Rough, Very Rough}. For clarification, the data structure used for training is written below:

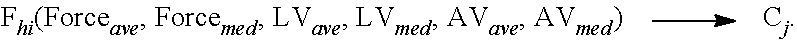

Finally, when testing the network (when given unseen/unlabeled data), it calculates the max (Cⱼ) corresponding to each path and assigns them to a qualitative deformation and texture class. For an unseen point that overlaps or is contained within multiple qualitative classes, the architecture involves the OCN and the CCN section to classify the input FV correctly with high accuracy and precision.

2) Time Series Classification-Learning Shapelets: Time series classification-Learning shapelets is the second algorithm adapted in the work. Implementing this approach requires the use of “shapelets”. A shapelet is a discriminative sub-sequence of time that best predicts a target variable. The distance between a shapelet and a time series is defined as the minimum of the distances between this shapelet and all the shapelets of identical length extracted from a time series [10]. The benefits of TSC-LS are that it can be trained using continuous data, which suits the purposes of our project. Moreover, shapelets are easily interpretable and compact, which leads to fast classification [11]. There are many methods for training shapelets, but the one adopted was created by Grabocka et al. [10]. The present inventors considered a new mathematical formalization to learn each shapelet via a classification objective function, and a tailored stochastic gradient learning algorithm was applied.

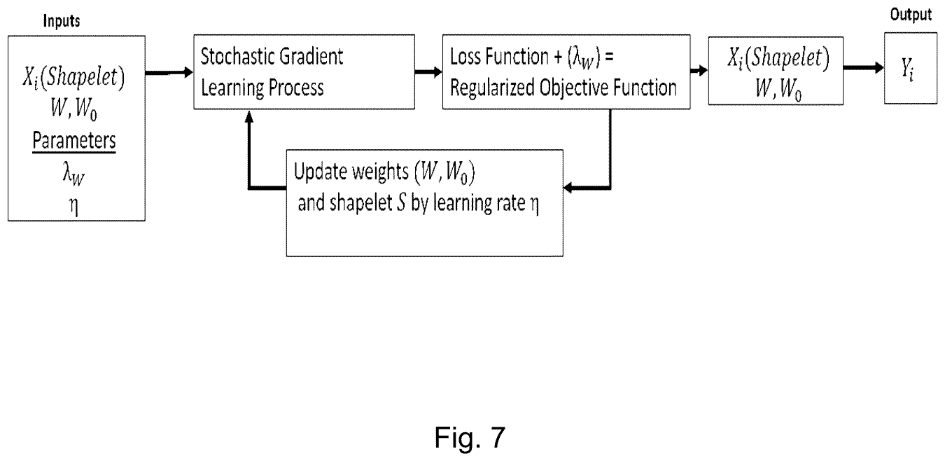

The flow chart displayed in FIG. 7 demonstrates the inputs and outputs of the TSC-LS. The inputs could be a univariate time series (UTS), where X=[x₁, x₂, . . . , x_{T}] is an ordered set of real values, and the number of real values T is equal to the length of X. The inputs could also be an M-dimensional multivariate time series (MTS), X=[X¹, X², . . . , X^{M}] consisting of M different univariate time series with Xⁱ∈R^{T}. The classification's output is a labeled vector Yⱼ of length K where each element j∈[1,K]. Given a dataset D=(X₁, Y₁), (X₂, Y₂), . . . , (X_{N}, Y_{N}) which is a collection of pairs (Xᵢ, Yⱼ), where Xᵢ is either a univariate or multivariate time series with a corresponding label Yⱼ; the algorithm assigns 1 to the corresponding label Yⱼ if the class of Xᵢ is j and 0 otherwise. The objective of the TSC-LS is to use the dataset D to train its classifier to map from the space of possible inputs to a probability distribution over the class variable values [31].

The training of the TSC-LS is non-linear and works similarly to a multilayer perceptron (MLP). For example, given a shapelet-S and four distinct constraints (a random weight W and a random bias Wo, a regularization term AW, and a learning rate n) as inputs. First, the algorithm goes through a learning process. Second, it calculates a regular object function based on the loss function and the regularization term. Third, the weights-W, Wo and shapelet-S are updated by a learning rate n. The training is done until the algorithm receives the desired shapelet and weights corresponding to the correct label Yᵢ.

In the case of the achieved system, two paths exiting the feature extraction section are examined; the deformation path inputs will consist of a multivariate time series data X=[Forceₐᵥₑ, Force_{med}, Displacementₐᵥₑ, Displacement_{med}] and output a corresponding class label Yⱼ. For deformation, the class label Yⱼ∈{Very Soft, Soft, Hard, VeryHard}. For clarification, the inputs and output are shown below:

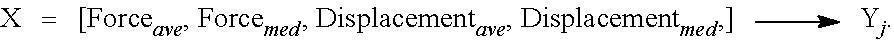

Simultaneously, the texture's path will have inputs X= [Forceₐᵥₑ, Force_{med}, LVₐᵥₑ, LV_{med}, AVₐᵥₑ, AV_{med}] that comprise a multivariate time series dataset. The output of the texture path will be a classification class label Yⱼ where Yⱼ∈{Very Smooth, Smooth, Rough, Very Rough}. For clarification, the data structure used for training is shown below:

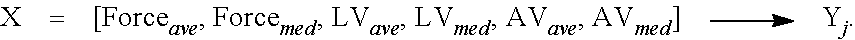

Lastly, when testing the algorithm, shapelets in each interval of an unseen MTS, X, are accurately associated with a previously learned shapelet. As a result, the TSC-LS classifies the unlearned shapelet to a class label Yⱼ, which is linked to a closely related learned shapelet.

### Example 6—Performance Evaluation

A. Experimental setup—The experimental setup involved collecting deformation and texture data using the designed tactile sensor. The data was processed using the various smoothing techniques discussed above and then used to train and test each of the machine learning models. The results from the tested data were collected and analyzed.

To begin, the experimentation setup to determine how effectively the tactile sensor captures the deformation of a structure consisted of using four items/labels that ranged from very soft to hard. Specifically, the items used were a playdough-like structure (soft), a silicone rubber structure (hard), and an eraser (very hard). The item deformation level was considered based on a subjective truth. The fourth label was when the tactile sensor was stationary. This label depicts when the sensor is not actively palpating during a robotic surgical procedure. The data for each label is collected by continuously palpating each object and collecting the force value from the FSR sensor and the change in the magnetic field from the magnetometer sensor. A total of 4000 samples for each item are collected at a 50 Hz sample rate and revived by Arduino Uno for analysis. Using Python programming, serial communication was completed with the Arduino Uno, and the serial data was filtered using the two aforementioned smoothing filters-Average filter and Median filter. The two filters output four total attributes for the machine learning models. The attributes being: Forceₐᵥₑ, Force_{med}, Displacementₐᵥₑ, Displacement_{med}. These four attributes are used to train and test the RFMN and the TSCLS algorithms.

Next, the experimentation structure used to validate the texture detection portion of the tactile sensor considers using four objects/labels that range from very smooth to rough. The objects used for experimentation were a glass surface (smooth), 120 medium drywall sandpaper (rough), and the side of a carrot grater (very rough). The fourth label was considered when the tactile sensor was not palpating a surface. Similar to the deformation experimental setup, these texture labels are based on a subjective truth. The data for each label is collected by continuously sliding the tactile sensor back and forth on the texture's surface and collecting the force signals from the FSR sensor. A total of 4000 data samples at a 50 Hz rate were collected by the Arduino Uno's ADC, SDA, and SCL pins for analysis. The Arduino Uno's serial communication was analyzed in Python, and signal filtering was done using the Average and Median filters. Filtering the data led to six total features acting as inputs to the machine learning models. The features were: Forceₐᵥₑ, Force_{med}, LVₐᵥₑ, LV_{med}, AVₐᵥₑ, AV_{med}. The six attributes are used for training and testing the RFMN and the TSC-LS algorithms.

B. Results and discussion-Analyzing the features for deformation and texture involves training and testing the RFMN algorithm and TSCLS classification. First, for the RFMN model, the theta expansion coefficient values considered are 0.2 and 0.3; for each theta, the gamma coefficient varies from 1 to 6. For each interval variation of theta and gamma, 10-fold cross-validation is executed. Lastly, the average accuracy, precision, mean-absolute-error (MAE), and mean-squared-error (MSE) metrics values for each fold are captured and reported in Table 1.

Similarly, for the TSC-LS model, the shapelet length (SL) varies from 1 to 4. For each shapelet length, two different types of distance measurement (DM) are considered: Euclidean and cosine. For each interval variation, 10-fold cross-validation is accomplished. Finally, the average accuracy, precision, mean-absolute-error (MAE), and mean-squared-error (MSE) metrics values got very fold and are captured and reported in Table 1.

TABLE 1
10-fold Cross-Validation Results

| Palpation | Algorithms | | | 10-fold Average Classification and Regression Metrics | | | |
|---|---|---|---|---|---|---|---|
| Types | Used | Parameters | | Accuracy | Precision | MAE | MSE |
| Deformation | RFMN | θ = 0.2 | \|γ = 1 | 76.42% | 76.42% | 39.68% | 81.89% |
| Analysis | | | γ = 2 | 77.11% | 77.11% | 38.23% | 78.94% |
| | | | γ = 3 | 77.20% | 77.20% | 38.03% | 78.51% |
| | | | γ = 4 | 77.23% | 77.23% | 37.97% | 78.36% |
| | | | γ = 5 | 77.25% | 77.25% | 37.93% | 78.30% |
| | | | γ = 6 | 77.18% | 77.18% | 38.11% | 78.72% |
| | | θ = 0.3 | γ = 1 | 82.77% | 82.77% | 19.41% | 23.76% |
| | | | γ = 2 | 82.85% | 82.85% | 19.23% | 23.39% |
| | | | γ = 3 | 82.88% | 82.88% | 19.16% | 23.22% |
| | | | γ = 4 | 82.88% | 82.88% | 19.15% | 23.20% |
| | | | γ = 5 | 82.36% | 82.36% | 20.39% | 25.88% |
| | | | γ = 6 | 78.35% | 78.35% | 28.08% | 40.94% |
| | TSC-LS | DM = | SL = 1 | 82.16% | 82.16% | 18.13% | 18.80% |
| | Multivariate | euclidean | SL = 2 | 82.99% | 82.99% | 17.04% | 17.10% |
| | | | SL = 3 | 83.66% | 83.66% | 16.35% | 16.38% |
| | | | SL = 4 | 83.81% | 83.81% | 16.19% | 16.19% |
| | | DM = | SL = 1 | 39.60% | 39.60% | 92.97% | 168.11% |
| | | cosine | SL = 2 | 39.73% | 39.73% | 92.77% | 167.72% |
| | | | SL = 3 | 39.45% | 39.45% | 90.65% | 160.84% |
| | | | SL = 4 | 44.69% | 44.69% | 80.49% | 135.95% |
| Texture | RFMN | θ = 0.2 | γ = 1 | 68.36% | 68.36% | 52.25% | 96.16% |
| Analysis | | | γ = 2 | 68.36% | 68.36% | 52.25% | 96.16% |
| | | | γ = 3 | 68.36% | 68.36% | 52.25% | 96.16% |
| | | | γ = 4 | 68.35% | 68.35% | 52.21% | 95.96% |
| | | | γ = 5 | 68.27% | 68.27% | 52.11% | 95.39% |
| | | | γ = 6 | 66.76% | 66.76% | 53.24% | 95.28% |
| | | θ = 0.3 | γ = 1 | 65.69% | 65.69% | 58.53% | 108.77% |
| | | | γ = 2 | 65.69% | 65.69% | 58.53% | 108.77% |
| | | | γ = 3 | 65.71% | 65.71% | 58.49% | 108.69% |
| | | | γ = 4 | 65.66% | 65.66% | 58.49% | 108.54% |
| | | | γ = 5 | 63.24% | 63.24% | 60.72% | 110.33% |
| | | | γ = 6 | 59.47% | 59.47% | 64.17% | 112.85% |
| | TSC-LS | DM = | SL = 1 | 61.61% | 61.61% | 60.36% | 106.57% |
| | Multivariate | euclidean | SL = 2 | 89.50% | 89.50% | 17.68% | 32.03% |
| | | | SL = 3 | 85.78% | 85.78% | 24.08% | 43.79% |
| | | | SL = 4 | 86.87% | 86.87% | 19.99% | 33.73% |
| | | DM = | SL = 1 | 27.61% | 27.61% | 124.53% | 106.57% |
| | | cosine | SL = 2 | 30.45% | 30.45% | 119.65% | 100% |
| | | | SL = 3 | 32.66% | 32.66% | 108.66% | 100% |
| | | | SL = 4 | 24.58% | 24.58% | 124.77% | 100% |

Depicted in Table 1 are all the results obtained from the experiment. The TSC-LS approach outperforms the RFMN by a minimal margin for deformation validation. The best metric values from TSC-LS classification are when the shapelet length is four and the distance measurement used is Euclidean. Meanwhile, the best metric values from the RFMN algorithm are when theta is 0.3 and gamma is 4. Similarly, the TSC-LS procedure outperformed the RFMN algorithm for texture validation. In this case, the optimal metric acquired from TSC-LS classification was when the shapelet length was two, and the distance measurement was Euclidean. In this case, the best metric values obtained from the RFMN algorithm were when theta was 0.2 and gamma was 1, 2 or 3. It should be noted that although the TSCLS classification outperformed the RFMN algorithm by a slight difference, a few considerations should be stated that could potentially alter the metric outcomes. Considerations include completion time, dimensionality addition/reduction, and additional data sampling collection. However, since the TSC-LS outperformed the RFMN in this data sample case, it would be best to use it during a robotic surgical application. Furthermore, the tactile sensor design used in this study effectively distinguishes between hardness and texture types, demonstrating high accuracies in training and testing machine learning methods with collected feature data.

The approach for tactile feedback presented here uses a state-of-the-art tactile sensor to capture two characteristics needed for organ or tissue palpation in RAMIS—specifically, identifying the softness and texture types of a biological structure when performing robotic surgery. The framework accomplishes this by extracting experimental data from the tactile sensor using a microcontroller and then processing the data as features for training and testing two machine learning algorithms—the RFMN and the TSC-LS classification. After training and testing the algorithms, results were considered based on comparing both algorithms. The TSC-LS classification proved superior to the RFMN algorithm when using 10-fold validation for the deformation and texture detection of various objects.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

### REFERENCES

[1] W. Othman and M. A. Qasaimeh, “Tactile sensing for minimally invasive surgery: Conventional methods and potential emerging tactile technologies,” Frontiers in Robotics and AI, p. 376, 2021.
[2] C. Huang, Q. Wang, M. Zhao, C. Chen, S. Pan, and M. Yuan, “Tactile perception technologies and their applications in minimally invasive surgery: a review,” Frontiers in Physiology, vol. 11, p. 611596, 2020.
[3] P. Puangmali, K. Althoefer, L. D. Seneviratne, D. Murphy, and P. Dasgupta, “State-of-the-art in force and tactile sensing for minimally invasive surgery,” IEEE Sensors Journal, vol. 8, no. 4, pp. 371-381, 2008.
[4] V. Vitiello, K.-W. Kwok, and G.-Z. Yang, “Introduction to robotassisted minimally invasive surgery (mis),” in Medical Robotics, pp. 1-P1, Elsevier, 2012.
[5] A. Menciassi, A. Eisinberg, M. C. Carrozza, and P. Dario, “Force sensing microinstrument for measuring tissue properties and pulse in microsurgery,” IEEE/ASME transactions on mechatronics, vol. 8, no. 1, pp. 10-17, 2003.
[6] “3D magnetometer micro-power and cost-conscious,” Melexis, https://www.melexis.com/en/product/MLX90392/3D-Magnetometermicro-power-and-cost-conscious (accessed Feb. 2, 2024).
[7] “LSM6DSOX,” STMicroelectronics, https://www.st.com/en/mems and-sensors/lsm6dsox.html (accessed Feb. 2, 2024).
[8] A. S. Naidu, R. V. Patel, and M. D. Naish, “Low-cost disposable tactile sensors for palpation in minimally invasive surgery,” IEEE/ASME Transactions on Mechatronics, vol. 22, no. 1, pp. 127-137, 2016.
[9] J. Grabocka, N. Schilling, M. Wistuba, and L. Schmidt-Thieme, “Learning time-series shapelets,” in Pro-ceedings of the 20th ACM SIGKDD international conference on Knowledge discovery and data mining, pp. 392-401, 2014.
[10] C. Ji, S. Liu, C. Yang, L. Pan, L. Wu, and X. Meng, “A shapelet selection algorithm for time series classifica-tion: New directions,” Procedia Computer Science, vol. 129, pp. 461-467, 2018.
[11] A. R. Lanfranco, A. E. Castellanos, J. P. Desai, and W. C. Meyers, “Robotic surgery: a current perspective,” Annals of surgery, vol. 239, no. 1, p. 14, 2004.
[12] B. Jaffray, “Minimally invasive surgery,” Archives of disease in childhood, vol. 90, no. 5, pp. 537-542, 2005.
[13] H. Liu, J. Greco, X. Song, J. Bimbo, L. Seneviratne, and K. Althoefer, “Tactile image based contact shape recognition using neural network,” in 2012 IEEE International Conference on Multisensor Fusion and Integration for Intelligent Systems (MFI), pp. 138-143, IEEE, 2012.
[14] D. Govalla and J. Rozenblit, “Towards a method to provide tactile feedback in minimally invasive robotic surgery,” in Computer Aided Systems Theory-EUROCAST 2022: 18th International Conference, Las Palmas de Gran Canaria, Spain, Feb. 20-25, 2022, Revised Selected Papers, pp. 496-503, Springer, 2023.
[15] A. L. Trejos, J. Jayender, M. Perri, M. D. Naish, R. V. Patel, and R. Malthaner, “Robot-assisted tactile sensing for minimally invasive tumor localization,” The International Journal of Robotics Research, vol. 28, no. 9, pp. 1118-1133, 2009.
[16] K. Li, B. Pan, J. Zhan, W. Gao, Y. Fu, and S. Wang, “Design and performance evaluation of a 3-axis force sensor for mispalpation,” Sensor Review, 2015.
[17] X. Wang, H. Zhang, L. Dong, X. Han, W. Du, J. Zhai, C. Pan, and Z. L. Wang, “Self-powered high-resolution and pressure-sensitive triboelectric sensor matrix for real-time tactile mapping,” Advanced materials, vol. 28, no. 15, pp. 2896-2903, 2016.
[18] T. Li, C. Shi, and H. Ren, “A high-sensitivity tactile sensor array based on fiber bragg grating sensing for tissue palpation in minimally invasive surgery,” IEEE/ASME transactions on mechatronics, vol. 23, no. 5, pp. 2306-2315, 2018.
[19] B. S. Zapata-Impata, P. Gil, and F. Torres, “Learning spatio temporal tactile features with a convlstm for the direction of slip detection,” Sensors, vol. 19, no. 3, p. 523, 2019.
[20] C. Pacchierotti, D. Prattichizzo, and K. J. Kuchenbecker, “Cutaneous feedback of fingertip deformation and vibration for palpation in robotic surgery,” IEEE Transactions on Biomedical Engineering, vol. 63, no. 2, pp. 278-287, 2015.
[21] A. V. Nandedkar and P. K. Biswas, “A general reflex fuzzy min-max neural network,” Eng. Lett., vol. 14, no. 1, pp. 195-205, 2007.
[22] P. Kozyr, A. Saveliev, and L. Kuznetsov, “Determining distance to an object and type of its material based on data of capacitive sensor signal and machine learning techniques,” in 2021 International Siberian Conference on Control and Communications (SIBCON), pp. 1-5, IEEE, 2021.
[23] J.-H. Lee and C.-H. Won, “The tactile sensation imaging system for embedded lesion characterization,” IEEE journal of biomedical and health informatics, vol. 17, no. 2, pp. 452-458, 2013.
[24] M. Sadeghi-Goughari and A. Mojra, “Intraoperative thermal imaging of brain tumors using a haptic-thermal robot with application in minimally invasive neurosurgery,” Applied Thermal Engineering, vol. 91, pp. 600-610, 2015.
[25] Z. Zhao, S. Voros, Y. Weng, F. Chang, and R. Li, “Tracking-by detection of surgical instruments in minimally invasive surgery via the convolutional neural network deep learning-based method,” Computer Assisted Surgery, vol. 22, no. sup1, pp. 26-35, 2017.
[26] Hart, Peter E., David G. Stork, and Richard O. Duda. Pattern classification. Hoboken: Wiley, 2000.
[27] Nandedkar, Abhijeet V., and Prabir K. Biswas. “Object recognition using reflex fuzzy min-max neural network with floating neurons.” Computer Vision, Graphics and Image Processing: 5th Indian Conference, ICVGIP 2006, Madurai, India, Dec. 13-16, 2006. Proceedings. Berlin, Heidelberg: Springer Berlin Heidelberg, 2006.
[28] P. K. Simpson, “Fuzzy min-max neural networks-part 1: Classification,” IEEE Trans. on Neural Networks, vol. 3, no. 5, pp. 776-786, 1992.
[29] E. Alhroob, M. F. Mohammed, C. P. Lim, and H. Tao, “A critical review on selected fuzzy min-max neural4 networks and their significance and challenges in pattern classification,” IEEE access, vol. 7, pp. 56129-56146, 2019.
[30] A. V. Nandedkar and P. K. Biswas, “Object recognition using reflex fuzzy min-max neural network with floating neurons,” in Computer Vision, Graphics and Image Processing, pp. 597-609, Springer, 2006.
[31] H. Ismail Fawaz, G. Forestier, J. Weber, L. Idoumghar, and P. A. Muller, “Deep learning for time series classification: a review,” Data mining and knowledge discovery, vol. 33, no. 4, pp. 917-963, 2019.

We claim:

1. A tactile sensing device comprising:
a) a distal end and a proximal end;
b) one or more micro-electromechanical system (MEMS) sensors disposed on the distal end of the device;
b) one or more force-sensitive resistor (FSR) sensors disposed on the distal end of the device; and
c) a microcontroller in communication with the one or more MEMS sensors and the one or more FSR sensors,
wherein, when the distal end of the device contacts a surface, the one or more MEMS sensors and the one or more FSR sensors are able to: measure two or more attributes of the distal end of the device as the distal end moves across or against the surface, generate electrical signals characterizing the measured attributes, and transmit the generated signals to the microcontroller, and
wherein the microcontroller is able to generate data of two or more characteristics of the surface from the transmitted signals, wherein the two or more characteristics of the surface comprise hardness, texture, vibrations, localized shape, slipperiness/stickiness, changes in consistency, and combinations thereof.

2. The tactile sensing device of claim 1, wherein the two or more characteristics of the surface comprise texture and hardness of the surface.

3. The tactile sensing device of claim 1 comprising two MEMS sensors disposed on the distal end of the device.

4. The tactile sensing device of claim 3, wherein the device is a surgical instrument capable of being operationally connected to a robotic surgical system.

5. The tactile sensing device of claim 1, wherein the one or more MEMS sensors and FSR sensors comprise a magnetometer sensor, an accelerometer sensor, a gyroscope sensor, or combinations thereof.

6. The tactile sensing device of claim 1, wherein the one or more MEMS sensors are able to measure changes in distance and position of the distal end of the device as the distal end is moved across the surface.

7. The tactile sensing device of claim 1, wherein the one or more FSR sensors are able to measure force applied to the distal end of the device when the distal end is pressed against the surface.

8. The tactile sensing device of claim 1, wherein the two or more measured attributes comprise force applied to the distal end, positional displacement of the distal end, linear vibration of the distal end, angular vibration of the distal end, and combinations thereof.

9. The tactile sensing device of claim 1, wherein the microcontroller comprises a processing unit able to execute a Reflex Fuzzy Min-Max Neural Network (RFMN) algorithm or a Time Series Classification-Learning Shapelets (TSC-LS) algorithm.

10. The tactile sensing device of claims claim 1, wherein the surface is biological tissue.

11. A method for determining the detecting two or more characteristics of a surface of an object comprising the steps of:
a) contacting the surface of the object with a tactile sensing device, wherein the tactile sensing device comprises one or more micro-electromechanical system (MEMS) sensors and one or more force-sensitive resistor (FSR) sensors disposed on a distal end of the tactile sensing device, and a microcontroller in communication with the one or more MEMS sensors and the one or more FSR sensors;
b) moving the distal end of the tactile sensing device across the surface of the object, pressing the distal end of the tactile sensing device against the surface of the object, or combinations thereof;
c) measuring two or more attributes of the distal end of the tactile sensing device as the distal end moves across or against the surface, generating electrical signals characterizing the measured attributes, and transmitting the generated signals to the microcontroller; and
d) generating data of the two or more characteristics object from the signals transmitted to the microcontroller, wherein the two or more characteristics of the surface comprise hardness, texture, vibrations, localized shape, slipperiness/stickiness, changes in consistency, and combinations thereof.

12. The method of claim 11, wherein the two or more characteristics of the surface comprise texture and hardness of the surface.

13. The method of claim 11, wherein the tactile sensing device is a surgical instrument and the method comprises operating the tactile sensing device using a robotic surgical system.

14. The method of claim 11, wherein the one or more MEMS sensors and FSR sensors comprise a magnetometer sensor, an accelerometer sensor, a gyroscope sensor, or combinations thereof.

15. The method of claim 11, wherein the object is biological tissue.

16. The method of claim 15, wherein the step of measuring two or more attributes comprises moving the distal end of the tactile sensing device across the surface of the biological tissue and measuring changes in distance and position of the distal end of the tactile sensing device as the distal end of the tactile sensing device is moved across the surface.

17. The method of claim 15, wherein the step of measuring two or more attributes comprises pressing the distal end of the tactile sensing device against the surface of the biological tissue, thereby generating a force against the distal end of the tactile sensing device, and measuring the force applied to the distal end of the tactile sensing device.

18. The method of claim 15, wherein the two or more characteristics of the surface comprise texture and hardness of the surface and wherein generating the texture and hardness data comprises identifying the texture and hardness of the biological tissue with recognized or known classifications.

19. The method of claim 18, wherein identifying the texture and hardness of the biological tissue comprises processing the signals transmitted to the microcontroller using a Reflex Fuzzy Min-Max Neural Network (RFMN) algorithm or a Time Series Classification-Learning Shapelets (TSC-LS) algorithm.

20. The method of claim 11, wherein the step of measuring two or more attributes comprises measuring the force applied to the distal end of the tactile sensing device, positional displacement of the distal end of the tactile sensing device, linear vibration of the distal end of the tactile sensing device, angular vibration of the distal end of the tactile sensing device, and combinations thereof.
